(19) United States
(12) Patent Application Publication
(10) Pub. No.: US 2005/0130951 A1
(43) Pub. Date: Jun. 16, 2005

(54) Superoxide dismutase mimics for the treatment of optic nerve and retinal damage

(75) Inventor: Peter G. Klimko, Fort Worth, TX (US)

Correspondence Address: Teresa J. Schultz;Alcon Research, Ltd., Mail Code Q-148, 6201 S. Freeway, Forth Worth, TX, 76134-2099, US

(73) Assignee: Alcon, Inc.

(21) Appl. No.: 11/000,213

(22) Filed: Nov. 30, 2004

Related U.S. Application Data
(60) Provisional application No. 60/528,830, filed on Dec. 11, 2003.

(51) Int. Cl. A61K 31/555

(52) U.S. Cl. 514/185

(57) ABSTRACT

Methods for preventing and treating damage to the optic nerve and/or retina by the use of SOD mimics, particularly pentaazacycle Mn^{(II)} complex SOD mimics, are disclosed.

This application claims priority from U.S. Ser. No. 60/528,830, filed Dec. 11, 2003.

### BACKGROUND OF THE INVENTION

1. Field of the Invention

This invention is directed to the treatment of optic nerve and retinal damage resulting from ischemia and hypoxia with compounds that are mimics of the enzyme superoxide dismutase.

2. Description of the Related Art

Retinal or optic nerve head damage, which can result in the loss of vision, can be caused by trauma and various pathological events including ischemia, hypoxia, or edema. There is increasing interest in pharmacological intervention using agents that treat instigators of the disease process, such as, nerve excitotoxicy or inappropriate oxygen consumption resulting from ischemia-reperfusion injury (see Clark 1999; David 1998; David 1997).

Many disease states are precipitated by periods of oxidative stress, such as occurs during ischemia-reperfusion injury. During these periods the body's natural defense mechanisms for dealing with toxic by-products of oxidative metabolism can be overwhelmed, leading to tissue damage from reactive oxygen species. One important component of this defense system is the superoxide dismutase (SOD) enzyme family. These enzymes contain a low valent metal (either Mn^{II} or a Cu^{I}/Zn^{I} binuclear linkage) which catalyze the disproportionation of the highly reactive superoxide radical anion to the less toxic entities O₂ and H₂O₂. If not quenched, the superoxide anion can (via its protonated form) abstract hydrogens from the allylic sites of fatty acids, leading to membrane damage. Additionally superoxide anion can react with NO, as shown, to produce peroxynitrite, a potent oxidizing agent which is believed to be an important player in the untoward biological effects of excessive NO production.

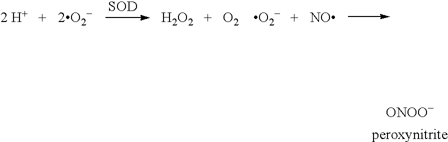

Literature from the ocular field suggests that SOD deficiency can lead to optic nerve damage, which can be rescued by SOD protein administration. In mice intraocularly injected with a ribozyme that selectively degraded SOD-2 mRNA, a loss of axons and myelin in the optic nervehead and retinal ganglion cells was observed (Qi et. al. 2003); SOD-2 is a Mn-containing superoxide dismutase, and is primarily expressed in mitochondria. In SOD-2^{-/-} mice a thinning of the optic nerve fiber layer and the optic nerve cross sectional area was noted in comparison to wild-type mice (Sandbach et. al. 2001). Intravitreal injections of SOD protein gave functional and histological protection in a rat ocular ischemia-reperfusion model; SOD was more efficacious than dimethylthiourea with respect to functional protection (99% vs. 40%) (Rios et. al. 1999).

Superoxide oxidation of serotonin to the neurotoxin tryptamine 4,5-dione (T-4,5-D) has been hypothesized to be important cerebral neuronal damage to due ischemia-reperfusion and to amphetamine exposure (Jiang and Dryhurst 2002; Wrona and Dryhurst 2001; Jiang et. al. 1999; Wrona and Dryhurst 1998). T-4,5-D uncouples mitochondrial respiration via irreversible inhibition of NADH-coenzyme Q1 reductase and cytochrome c oxidase, and also irreversibly inactivates tryptophan hydroxylase.

The use of intravenously dosed Mn SOD itself to treat or prevent oxidative stress-related tissue injury in humans, such as tissue damage due to cerebral or myocardial ischemia-reperfusion injury, has been unsuccessful due to bioavailability and immunogenic issues. These problems are thought to be due to the fact that Mn SOD is a high molecular weight species. A low molecular weight compound that catalyzes superoxide disproportionation with efficiency comparable to endogenous Mn SOD could be a good candidate for minimizing the aforementioned side effects. Salvemini et al. have disclosed a class of Mn(II)-pentaaza macrocycle complexes as low molecular weight SOD mimics. For example, in a rat model of intestinal ischemia-reperfusion, 90% of animals dosed with 1 mg/kg of compound 1 survived after 4 h, compared to 0% survival for untreated animals (Salvemini et. al. 1999); WO 98/58636; Salvemini et. al. 2000). Compound 1 has also been shown to inhibit NMDA-induced cell death in a is mixed neuronal/glial forebrain cell culture (Salvemini et. al. 2002a), and to improve survival time, reduce tissue damage, and reduce the production of the inflammatory markers ICAM-1, P-selectin, and nitrotyrosine, in a rat model of mycardial ischemia-reperfusion injury (Salvemini et. al. 2002). These compounds have also been disclosed for enhancing the stability of implanted biopolymeric prosthetic devices (including ocular implants—WO 00/72893 A2) and for the treatment of pain (U.S. Pat. Nos. 6,180,620 B1 and 6,214,817B1).

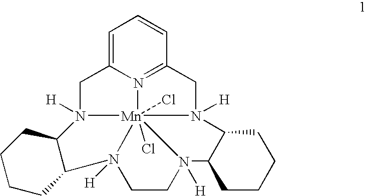

The use of certain Mn-salen complexes as SOD and catalase mimics with therapeutic activity has also been disclosed. For example, compound 2 has been shown to be neuroprotective in a rat stroke model (Baker et. al. 1998; Doctrow et. al. 2002), while compound 3 was found to increase the lifespan of mice that were deficient in endogenous expression of SOD-2 (Melov et. al. 2001).

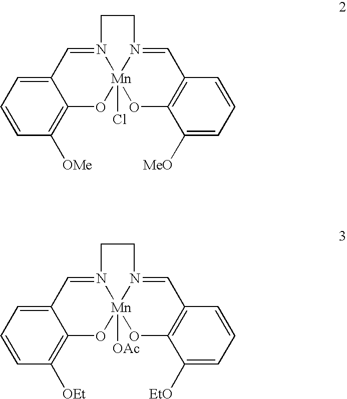

Other investigators have reported the use of antioxidant compounds to treat ocular diseases. Crapo et. al., have disclosed the use of porphyrin-containing SOD mimics for treating glaucoma and macular degeneration (U.S. Pat. Nos. 5,994,339 and 6,127,356). Campbell et al. have disclosed the use of certain bipyridyl Mn(II or III)phenolate complexes for treating free-radical associated diseases (U.S. Pat. No. 6,177,419 B1). Levin has disclosed the use of carvedilol and its derivatives and metabolites as scavengers of ROS to reduce retinal ganglion cell death (WO 00/07584 A2). Brownlee has disclosed the use of a manganese tetrakis(benzoic acid) porphyrin for reducing ROS accumulation under high glucose conditions for treating diabetic retinopathy (WO 00/19993 A2). The stable free radical 4-hydroxy-2,2,6,6-tetramethylpiperidine-1-oxyl, a metal-free SOD mimic, has been reported to inhibited light-induced retinal damage in albino rats (Wang et. al. 1995). However, in none of these reports were the compounds of the present invention disclosed or suggested for the treatment of optic nerve and retinal damage.

### SUMMARY OF THE INVENTION

The present invention overcomes these and other drawbacks of the prior art by providing methods to treat persons suffering from chronic or acute optic nerve and/or retinal damage. This application is directed to the use of certain mimics of the enzyme superoxide dismutase to treat persons suffering from chronic or acute optic nerve and/or retinal damage. The present invention discloses compositions and methods for systemic, topical, and intraocular administration of at least one SOD mimic in an amount effective to prevent or to treat retinal and/or optic nerve head tissue damage.

### DETAILED DESCRIPTION OF THE INVENTION

It has now been discovered that certain SOD mimics are useful for the treatment of patients suffering from chronic or acute optic nerve and/or retinal damage. These compounds are of formula I: 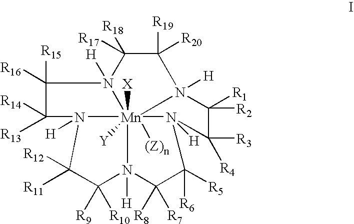
wherein:
R¹⁻²⁰ are independently H, alkyl, alkenyl, alkynyl, cycloalkyl, cycloalkenyl, aryl, heteroaryl, heterocycloalkyl, or heterocycloalkenyl, each of which is optionally substituted with an alkyl, alkenyl, alkynyl, cycloalkyl, cycloalkenyl, aryl, heteroaryl, heterocycloalkyl, heterocycloalkenyl, halo, trihalomethyl, acyl, alkoxycarbonyl, alkylsulfonyl, or arylsulfonyl group, or a free or functionally modified hydroxyl, amino, or thiol group;
or two of the R groups on the same (e.g., R¹ and R², or R³ and R⁴, or R⁵ and R⁶, etc.) or adjacent (e.g., R¹ and R³, or R³ and R⁵, or R⁶ and R⁷, etc.) sites, together with the carbon atoms to which they are attached, form an optionally unsaturated or aromatic C₃₋₂₀ carbocycle, the carbocycle being optionally substituted with alkyl, alkenyl, alkynyl, cycloalkyl, cycloalkenyl, aryl, heteroaryl, heterocycloalkyl, heterocycloalkenyl, halo, trihalomethyl, acyl, alkoxycarbonyl, alkylsulfonyl, or arylsulfonyl group, or a free or functionally modified hydroxyl, amino, or thiol group;
or two of the R groups on the same (e.g., R¹ and R², or R³ and R⁴, or R⁵ and R⁶, etc.) or adjacent (e.g., R¹ and R³, or R³ and R⁵, or R⁶ and R⁷, etc.) sites, together with the carbon atoms to which they are attached, form an optionally unsaturated or aromatic C₂₋₂₀ nitrogen-containing heterocycle, the heterocycle being optionally substituted optionally substituted with alkyl, alkenyl, alkynyl, cycloalkyl, cycloalkenyl, aryl, heteroaryl, heterocycloalkyl, heterocycloalkenyl, halo, trihalomethyl, acyl, alkoxycarbonyl, alkylsulfonyl, or arylsulfonyl group, or a free or functionally modified hydroxyl, amino, or thiol group;
it being understood that in all cases the nitrogens binding the Mn center in the drawing for I will lack hydrogens when the nitrogen is already trisubstituted (e.g., when the relevant nitrogen is part of a pyridine ring);
X, Y, and Z are pharmaceutically acceptable anions; and
n is 0-3.

Compounds I of the present invention are known, and their syntheses are disclosed in U.S. Pat. No. 6,214,817 B1, which is herein incorporated by reference.

As used herein, the terms “pharmaceutically acceptable anion” means any anion that would be suitable for therapeutic administration to a patient by any conventional means without significant deleterious health consequences. Examples of preferred pharmaceutically acceptable anions include chloride, bromide, acetate, benzoate, maleate, fumarate, and succinate.

The term “free hydroxy group” means an OH. The term “functionally modified hydroxy group” means an OH which has been functionalized to form: an ether, in which an alkyl group is substituted for the hydrogen; an ester, in which an acyl group is substituted for the hydrogen; a carbamate, in which an aminocarbonyl group is substituted for the hydrogen; or a carbonate, in which an alkoxycarbonyl group is substituted for the hydrogen. Examples of preferred groups include OH, OC(O)CH₃, OCH₃, OPh, OCH₂Ph, and OC(O)Ph.

The term “free amino group” means an N₂. The term “functionally modified amino group” means an NH₂ which has been functionalized to form: an alkoxyamino or hydroxyamino group, in which an alkoxy or hydroxy group is substituted for one of the hydrogens; an alkylamino group, in which an alkyl group is substituted for one or both of the hydrogens; an amide, in which an acyl group is substituted for one of the hydrogens; a carbamate, in which an alkoxycarbonyl group is substituted for one of the hydrogens; or a urea, in which an aminocarbonyl group is substituted for one of the hydrogens. Combinations of these substitution patterns, for example an NH₂ in which one of the hydrogens is replaced by an alkyl group and the other hydrogen is replaced by an alkoxycarbonyl group, also fall under the definition of a functionally modified amino group and are included within the scope of the present invention. Examples of preferred groups include NH₂, NHCH₃, N(CH₃)₂, NHPh, NHC(O)Ph, NHC(O)CH₃, NHC(O)OCH₃, and NHC(O)OPh.

The term “free thiol group” means an SH. The term “functionally modified thiol group” means an SH which has been functionalized to form: a thioether, where an alkyl, aryl, cycloalkyl, heterocycloalkyl, alkenyl, cycloalkenyl, heterocycloalkenyl, alkynyl, or heteroaryl group is substituted for the hydrogen; or a thioester, in which an acyl group is substituted for the hydrogen. Examples of preferred moieties include SH, SPh, SC(O)CH₃, SCH₃, SC₂H₅, SC(CH₃)₃, S-cyclohexyl, SCH₂CO₂CH₃, SCH₂CO₂C₂H₅, SCH₂C(O)C₂H₅, and SCH₂C(O)CH₃.

The term “acyl” represents a group that is linked by a carbon atom that has a double bond to an oxygen atom and a single bond to another carbon atom.

The term “alkyl” includes straight or branched chain aliphatic hydrocarbon groups that are saturated and have 1 to 15 carbon atoms. The alkyl groups may be substituted with other groups, such as halogen, hydroxyl or alkoxy. Preferred straight or branched alkyl groups include methyl, ethyl, propyl, isopropyl, butyl and t-butyl.

The term “cycloalkyl” includes straight or branched chain, saturated or unsaturated aliphatic hydrocarbon groups which connect to form one or more rings, which can be fused or isolated. The rings may be substituted with other groups, such as halogen, hydroxyl, alkoxy, or lower alkyl. Preferred cycloalkyl groups include cyclopropyl, cyclobutyl, cyclopentyl and cyclohexyl.

The term “alkenyl” includes straight or branched chain hydrocarbon groups having 1 to 15 carbon atoms with at least one carbon-carbon double bond. The chain hydrogens may be substituted with other groups, such as halogen. Preferred straight or branched alkeny groups include, allyl, 1-butenyl, 1-methyl-2-propenyl and 4-pentenyl.

The term “cycloalkenyl” includes straight or branched chain, saturated or unsaturated aliphatic hydrocarbon groups which connect to form one or more non-aromatic rings containing a carbon-carbon double bond, which can be fused or isolated. The rings may be substituted with other groups, such as halogen, hydroxyl, alkoxy, or lower alkyl. Preferred cycloalkenyl groups include cyclopentenyl and cyclohexenyl.

The term “alkoxy” represents an alkyl group attached through an oxygen linkage.

The term “carbonyl group” represents a carbon atom double bonded to an oxygen atom, wherein the carbon atom has two free valencies.

The term “alkoxycarbonyl” represents an alkoxy group bonded from its oxygen atom to the carbon of a carbonyl group, the carbonyl group itself being bonded to another atom through its carbon atom.

The term “aminocarbonyl” represents an amino group bonded from its nitrogen atom to the carbon atom of a carbonyl group, the carbonyl group itself being bonded to another atom through its carbon atom.

The term “lower alkyl” represents alkyl groups containing one to six carbons (C₁-C₆).

The term “halogen” represents fluoro, chloro, bromo, or iodo.

The term “aryl” refers to carbon-based rings which are aromatic. The rings may be isolated, such as phenyl, or fused, such as naphthyl. The ring hydrogens may be substituted with other groups, such as lower alkyl, or halogen.

The term “heteroaryl” refers to aromatic hydrocarbon rings which contain at least one heteroatom such as O, S, or N in the ring. Heteroaryl rings may be isolated, with 5 to 6 ring atoms, or fused, with 8 to 10 atoms. The heteroaryl ring(s) hydrogens or heteroatoms with open valency may be substituted with other groups, such as lower alkyl or halogen. Examples of heteroaryl groups include imidazole, pyridine, indole, quinoline, furan, thiophene, pyrrole, tetrahydroquinoline, dihydrobenzofuran, and dihydrobenzindole.

Preferred compounds of the present invention include those of formula I, wherein:
R⁷R⁸C—N—CR⁹R¹⁰ forms a 5-8 membered saturated or unsaturated (including aromatic) ring, the ring being optionally substituted with alkyl, alkenyl, alkynyl, cycloalkyl, cycloalkenyl, aryl, heteroaryl, heterocycloalkyl, heterocycloalkenyl, halo, trihalomethyl, acyl, alkoxycarbonyl, alkylsulfonyl, or arylsulfonyl group, or a free or functionally modified hydroxyl, amino, or thiol group;
R⁵, R⁶, R¹¹, R¹², R¹⁷, R¹⁸, R¹⁹, and R²⁰ are the same or different and are H or alkyl;
R¹R²C—CR³R⁴ and R¹³R¹⁴C—CR¹⁵R¹⁶ are the same or different and form a 5-8 membered saturated or unsaturated (including aromatic) ring, the ring being optionally substituted with alkyl, alkenyl, alkynyl, cycloalkyl, cycloalkenyl, aryl, heteroaryl, heterocycloalkyl, heterocycloalkenyl, halo, trihalomethyl, acyl, alkoxycarbonyl, alkylsulfonyl, or arylsulfonyl group, or a free or functionally modified hydroxyl, amino, or thiol group;
X and Y are chloride; and
n is 0.

The most preferred compounds of the present invention include the following:

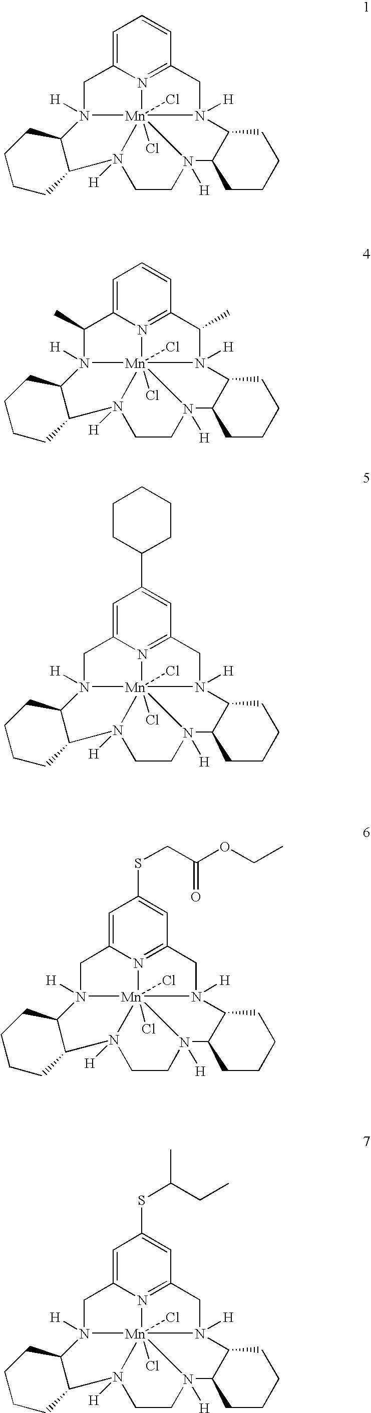

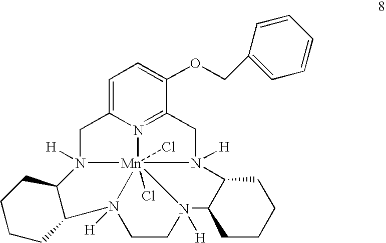

The syntheses of these compounds is disclosed in U.S. Pat. No. 6,214,817 B1.

The SOD mimics may be contained in various types of pharmaceutical compositions, in accordance with formulation techniques known to those skilled in the art. For example, the compounds may be included in tablets, capsules, solutions, suspensions, and other dosage forms adapted for oral administration; solutions and suspensions adapted for parenteral use; and solutions and suspensions adapted for topical ophthalmic, depot, or intra-ocular injection. Solutions, suspensions, and other dosage forms adapted for depot, oral, intra-ocular injection, and topical ophthalmic administration, such as eye drops or tissue irrigating solutions, are particularly preferred for the prevention or treatment of acute or chronic retinal or optic nerve head damage. Compositions can also be delivered topically to the eye according to the teachings in U.S. Pat. No. 5,952,378, which is herein incorporated by reference.

It is believed that compounds of Formula I are effective in preventing or treating damage to the retina and optic nerve, particularly damage resulting from ischemic or hypoxic stress. The compounds are also useful for treating damage arising from the presence of cyto or neurotoxic entities, such as glutamate and other excitatory amino acids or peptides, excess intracellular calcium, and free radicals. In particular, the compounds can be useful in treating damage associated with branch and central vein/artery occlusion, anterior ischemic optic neuropathy, trauma, edema, angle-closure glaucoma, open-angle glaucoma, retinitis pigmentosa (RP), retinal detachments, damage associated with laser therapy, including photodynamic therapy (PDT), and surgical light induced iatrogenic retinopathy. The compounds may also be used as an adjunct to ophthalmic surgery, such as, by vitreal or subconjunctival injection following surgery. The compounds may also be used to treat acute conditions or prophylactically, especially prior to surgery or non-invasive procedures.

The present invention is also directed to the provision of compositions adapted for treatment of retinal and optic nerve head tissues. The ophthalmic compositions of the present invention will include one or more SOD mimics and a pharmaceutically acceptable vehicle. Various types of vehicles may be used. The vehicles will generally be aqueous in nature. Aqueous solutions are generally preferred, based on ease of formulation, as well as a patient's ability to easily administer such compositions by means of instilling one to two drops of the solutions in the affected eyes. However, the SOD mimics of the present invention may also be readily incorporated into other types of compositions, such as suspensions, viscous or semi-viscous gels, or other types of solid or semi-solid compositions. Suspensions may be preferred for SOD mimics that are relatively insoluble in water. The ophthalmic compositions of the present invention may also include various other ingredients, such as buffers, preservatives, co-solvents, and viscosity building agents.

An appropriate buffer system (e.g., sodium phosphate, sodium acetate or sodium borate) may be added to prevent pH drift under storage conditions.

Ophthalmic products are typically packaged in multidose form. Preservatives are thus required to prevent microbial contamination during use. Suitable preservatives include: benzalkonium chloride, thimerosal, chlorobutanol, methyl paraben, propyl paraben, phenylethyl alcohol, edetate disodium, sorbic acid, polyquatemium-1, or other agents known to those skilled in the art. Such preservatives are typically employed at a level of from 0.001 to 1.0% weight/volume (“% w/v”).

The route of administration (e.g., topical, ocular injection, parenteral, or oral) and the dosage regimen will be determined by skilled clinicians, based on factors such as the exact nature of the condition being treated, the severity of the condition, and the age and general physical condition of the patient.

In general, the doses used for the above described purposes will vary, but will be in an effective amount to prevent, reduce or ameliorate retinal or optic nerve head tissue damage resulting from any of the above listed conditions. As used herein, the term “pharmaceutically effective amount” refers to an amount of one or SOD mimics of the present invention which will prevent, reduce, or ameliorate chronic or acute retinal or optic nerve head damage resulting from ischemic or hypoxic conditions in a human patient. The doses used for any of the above-described purposes will generally be from about 0.01 to about 100 milligrams per kilogram of body weight (mg/kg), administered one to four times per day. When the compositions are dosed topically, they will generally be in a concentration range of from 0.001 to about 5% w/v, with 1-2 drops administered 1-4 times per day.

When the SOD mimics of the present invention are administered during intraocular surgical procedures, such as through retrobulbar or periocular injection and intraocular perfusion or injection, the use of balanced salt irrigating solutions as vehicles are most preferred. BSS® Sterile Irrigating Solution and BSS Plus® Sterile Intraocular Irrigating Solution (Alcon Laboratories, Inc., Fort Worth, Tex., USA) are examples of physiologically balanced intraocular irrigating solutions. The latter type of solution is described in U.S. Pat. No. 4,550,022, the entire contents of which are hereby incorporated in the present specification by reference. Retrobulbar and periocular injections are known to those skilled in the art and are described in numerous publications including, for example, in Ophthalmic Surgery: Principles of Practice (1990).

As used herein, the term “pharmaceutically acceptable carrier” refers to any formulation that is safe, and provides the appropriate delivery for the desired route of administration of an effective amount of at least one compound of the present invention.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

The following Examples 1-2 are formulations useful for intraocular, periocular, or retrobulbar injection or perfusion.

### EXAMPLE 1

| Component | % w/v |
|---|---|
| Compound of formula I | 0.1 |
| Dibasic sodium phosphate | 0.2 |
| HPMC | 0.5 |
| Polysorbate 80 | 0.05 |
| Benzalkonium chloride | 0.01 |
| Sodium chloride | 0.75 |
| Edetate disodium | 0.01 |
| NaOH/HCI | q.s. to pH 7.4 |
| Purified water | q.s. to 100% |

### EXAMPLE 2

| Component | % w/v |
|---|---|
| Compound of formula I | 0.1 |
| Cremophor EL | 10 |
| Tromethamine | 0.12 |
| Boric acid | 0.3 |
| Mannitol | 4.6 |
| Edetate disodium | 0.1 |
| Benzalkonium chloride | 0.1 |
| NaOH/HCI | q.s. to pH 7.4 |
| Purified water | q.s. to 100% |

### EXAMPLE 3

The following tablet formulation can be made pursuant to U.S. Pat. No. 5,049,586, incorporated herein by reference.

| Component | % w/v |
|---|---|
| Compound of formula I | 60 |
| Magnesium oxide | 20 |
| Corn starch | 15 |
| Polyvinylpyrrolidone | 3 |
| Sodium carboxymethylcellulose | 1 |
| Magnesium stearate | 0.8 |

An SOD mimic of the present invention can be formulated in an ocular irrigating solution used during ophthalmic surgery to treat retinal or optic nerve head damage resulting from trauma due to injury or prevent damages resulting from the invasive nature of the surgery. The concentration of the SOD mimic in the irrigating solution will range from 0.001 to 5% w/v.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and structurally related may be substituted for the agents described herein to achieve similar results. All such substitutions and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

United States Patents
U.S. Pat. No. 4,550,022
U.S. Pat. No. 5,049,586
U.S. Pat. No. 5,952,378
U.S. Pat. No. 5,994,339
U.S. Pat. No. 6,127,356
U.S. Pat. No. 6,177,419 B1
U.S. Pat. No. 6,180,620 B1
U.S. Pat. No. 6,214,817 B1
WO 98/58636
WO 00/07584
WO 00/19993 A2
WO 00/72893

Books
OPHTHALMIC SURGERY: PRINCIPLES OF PRACTICE, Ed., G. L. Spaeth, W. B. Sanders Co., Philadelphia, Pa., U.S.A., pp. 85-87 (1990).

Other Publications
Baker et al., J. PHARMACOL. EXP. THER. 284: 215-221 (1998).
Clark, A F, “Current trends in antiglaucoma therapy,” EMERGING DRUGS 4: 333 (1999).
David, R, “Neuroprotection of the optic nerve in glaucoma,” ACTA OPHTHALMOL. SCAND. 75: 364 (1997).
David, R, “Changing therapeutic paradigms in glaucoma management,” EXPERT OPIN. INVEST. DRUGS 7: 1063 (1998).
Doctrow et al., J. MED. CHEM. 45: 4549-4558 (2002).
Jiang et al., CHEM. RES. TOXICOL. 12: 429-436 (1999).
Jiang and Dryhurst, CHEM. RES. TOXICOL. 15: 1242-1247 (2002).
Melov et al., J. NEUROSCI., 21: 8348-8353 (2001).
Qi et al., OPHTHALMOL. VIS. SCI. 44: 1088 (2003).
Rios et al., J. OCUL. PHARMACOL. THER. 15(6): 547 (1999).
Salvemini, D et al., SCIENCE 286: 304-306 (1999).
Salvemini, D et al., DRUGS FUTURE 25 (10): 1027 (2000).
Salvemini, D et al., J. PHARMACOL. EXP. THER. 301: 478 (2002a).
Salvemini, D et al., BR. J. PHARMACOL. 136 (6): 905 (2002b).
Sandbach et al., INVEST. OPHTHALMOL. VIS. SCI. 42: 2173 (2001).
Wang et al., RES. COMMUN. MOL. PATHOL. PHARMACOL. 89: 291-305 (1995).
Wrona and Dryhurst, CHEM. RES. TOXICOL. 11: 639-650 (1998).
Wrona and Dryhurst, CHEM. RES. TOXICOL. 14: 1184-1192 (2001).

1. A method for preventing damage to the optic nerve head or retina which comprises administering a pharmaceutically effective amount of superoxide dismutase mimic of formula I: 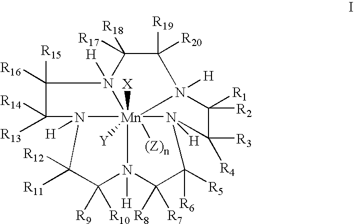
wherein:
R¹⁻²⁰ are independently H, alkyl, alkenyl, alkynyl, cycloalkyl, cycloalkenyl, aryl, heteroaryl, heterocycloalkyl, or heterocycloalkenyl, each of which is optionally substituted with an alkyl, alkenyl, alkynyl, cycloalkyl, cycloalkenyl, aryl, heteroaryl, heterocycloalkyl, heterocycloalkenyl, halo, trihalomethyl, acyl, alkoxycarbonyl, alkylsulfonyl, or arylsulfonyl group, or a free or functionally modified hydroxyl, amino, or thiol group;
or two of the R groups on the same (e.g., R¹ and R², or R³ and R⁴, or R⁵ and R⁶, etc.) or adjacent (e.g., R¹ and R³, or R³ and R⁵, or R⁶ and R⁷, etc.) sites, together with the carbon atoms to which they are attached, form an optionally unsaturated or aromatic C₃₋₂₀ carbocycle, the carbocycle being optionally substituted with alkyl, alkenyl, alkynyl, cycloalkyl, cycloalkenyl, aryl, heteroaryl, heterocycloalkyl, heterocycloalkenyl, halo, trihalomethyl, acyl, alkoxycarbonyl, alkylsulfonyl, or arylsulfonyl group, or a free or functionally modified hydroxyl, amino, or thiol group;
or two of the R groups on the same (e.g., R¹ and R², or R³ and R⁴, or R⁵ and R⁶, etc.) or adjacent (e.g., R¹ and R³, or R³ and R⁵, or R⁶ and R⁷, etc.) sites, together with the carbon atoms to which they are attached, form an optionally unsaturated or aromatic C₂₋₂₀ nitrogen-containing heterocycle, the heterocycle being optionally substituted optionally substituted with alkyl, alkenyl, alkynyl, cycloalkyl, cycloalkenyl, aryl, heteroaryl, heterocycloalkyl, heterocycloalkenyl, halo, trihalomethyl, acyl, alkoxycarbonyl, alkylsulfonyl, or arylsulfonyl group, or a free or functionally modified hydroxyl, amino, or thiol group;
it being understood that in all cases the nitrogens binding the Mn center in the drawing for I will lack hydrogens when the nitrogen is already trisubstituted (e.g., when the relevant nitrogen is part of a pyridine ring);
X, Y, and Z are pharmaceutically acceptable anions; and
n is 0-3.

2. The method of claim 1 wherein the damage is the result of ischemia and/or hypoxia.

3. The method of claim 2, wherein the damage is associated with a condition selected from the group consisting of branch and central vein/artery occlusion, angle-closure glaucoma, open-angle glaucoma, anterior ischemic optic neuropathy, RP, retinal detachments, laser therapy, and surgical light induced iatrogenic retinopathy.

4. The method of claim 1, wherein for the compound of formula I:
R⁷R⁸C—N—CR⁹R¹⁰ forms a 5-8 membered saturated or unsaturated (including aromatic) ring, the ring being optionally substituted with alkyl, alkenyl, alkynyl, cycloalkyl, cycloalkenyl, aryl, heteroaryl, heterocycloalkyl, heterocycloalkenyl, halo, trihalomethyl, acyl, alkoxycarbonyl, alkylsulfonyl, or arylsulfonyl group, or a free or functionally modified hydroxyl, amino, or thiol group;
R⁵, R⁶, R¹¹, R¹², R¹⁷, R¹⁸, R¹⁹, and R²⁰ are the same or different and are H or alkyl;
R¹R²C—CR³R⁴ and R¹³R¹⁴C—CR¹⁵R¹⁶ are the same or different and form a 5-8 membered saturated or unsaturated (including aromatic) ring, the ring being optionally substituted with alkyl, alkenyl, alkynyl, cycloalkyl, cycloalkenyl, aryl, heteroaryl, heterocycloalkyl, heterocycloalkenyl, halo, trihalomethyl, acyl, alkoxycarbonyl, alkylsulfonyl, or arylsulfonyl group, or a free or functionally modified hydroxyl, amino, or thiol group;
X and Y are chloride; and
n is 0.

5. The method of claim 4, wherein the compound is selected from the group consisting of:

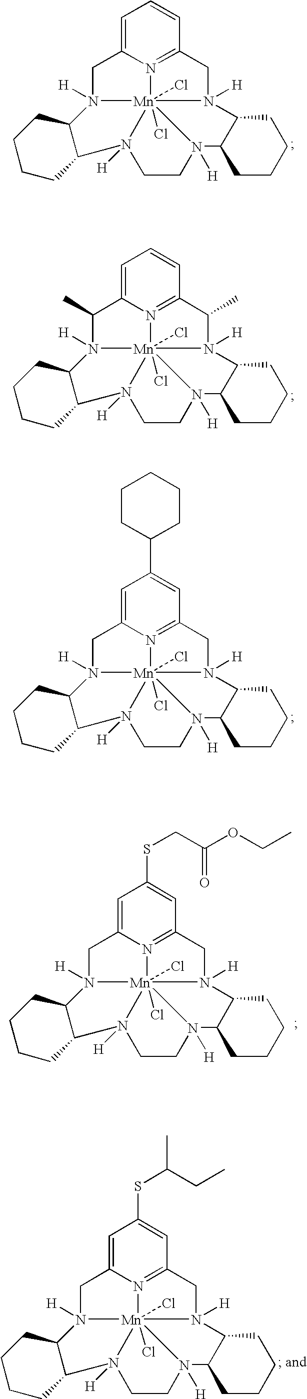

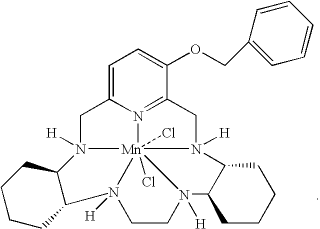

6. A composition for preventing damage to the optic nerve head or retina which comprises administering a pharmaceutically effective amount of superoxide dismutase mimic of formula I: 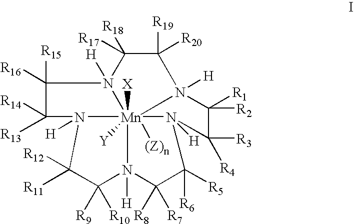
wherein:
R¹⁻²⁰ are independently H, alkyl, alkenyl, alkynyl, cycloalkyl, cycloalkenyl, aryl, heteroaryl, heterocycloalkyl, or heterocycloalkenyl, each of which is optionally substituted with an alkyl, alkenyl, alkynyl, cycloalkyl, cycloalkenyl, aryl, heteroaryl, heterocycloalkyl, heterocycloalkenyl, halo, trihalomethyl, acyl, alkoxycarbonyl, alkylsulfonyl, or arylsulfonyl group, or a free or functionally modified hydroxyl, amino, or thiol group;
or two of the R groups on the same (e.g., R¹ and R², or R³ and R⁴, or R⁵ and R⁶, etc.) or adjacent (e.g., R¹ and R³, or R³ and R⁵, or R⁶ and R⁷, etc.) sites, together with the carbon atoms to which they are attached, form an optionally unsaturated or aromatic C₃₋₂₀ carbocycle, the carbocycle being optionally substituted with alkyl, alkenyl, alkynyl, cycloalkyl, cycloalkenyl, aryl, heteroaryl, heterocycloalkyl, heterocycloalkenyl, halo, trihalomethyl, acyl, alkoxycarbonyl, alkylsulfonyl, or arylsulfonyl group, or a free or functionally modified hydroxyl, amino, or thiol group;
or two of the R groups on the same (e.g., R¹ and R², or R³ and R⁴, or R⁵ and R⁶, etc.) or adjacent (e.g., R¹ and R³, or R³ and R⁵, or R⁶ and R⁷, etc.) sites, together with the carbon atoms to which they are attached, form an optionally unsaturated or aromatic C₂₋₂₀ nitrogen-containing heterocycle, the heterocycle being optionally substituted optionally substituted with alkyl, alkenyl, alkynyl, cycloalkyl, cycloalkenyl, aryl, heteroaryl, heterocycloalkyl, heterocycloalkenyl, halo, trihalomethyl, acyl, alkoxycarbonyl, alkylsulfonyl, or arylsulfonyl group, or a free or functionally modified hydroxyl, amino, or thiol group;
it being understood that in all cases the nitrogens binding the Mn center in the drawing for I will lack hydrogens when the nitrogen is already trisubstituted (e.g., when the relevant nitrogen is part of a pyridine ring);
X, Y, and Z are pharmaceutically acceptable anions; and
n is 0-3,
and a pharmaceutically acceptable excipient.

7. The composition of claim 6, wherein for the compound of formula I:
R⁷R⁸C—N—CR⁹R¹⁰ forms a 5-8 membered saturated or unsaturated (including aromatic) ring, the ring being optionally substituted with alkyl, alkenyl, alkynyl, cycloalkyl, cycloalkenyl, aryl, heteroaryl, heterocycloalkyl, heterocycloalkenyl, halo, trihalomethyl, acyl, alkoxycarbonyl, alkylsulfonyl, or arylsulfonyl group, or a free or functionally modified hydroxyl, amino, or thiol group;
R⁵, R⁶, R¹¹, R¹², R¹⁷, R¹⁸, R¹⁹, and R²⁰ are the same or different and are H or alkyl;
R¹R²C—CR³R⁴ and R¹³R¹⁴C—CR¹⁵R¹⁶ are the same or different and form a 5-8 membered saturated or unsaturated (including aromatic) ring, the ring being optionally substituted with alkyl, alkenyl, alkynyl, cycloalkyl, cycloalkenyl, aryl, heteroaryl, heterocycloalkyl, heterocycloalkenyl, halo, trihalomethyl, acyl, alkoxycarbonyl, alkylsulfonyl, or arylsulfonyl group, or a free or functionally modified hydroxyl, amino, or thiol group;
X and Y are chloride; and
n is 0.

8. The composition of claim 7, wherein the compound is selected from the group consisting of:

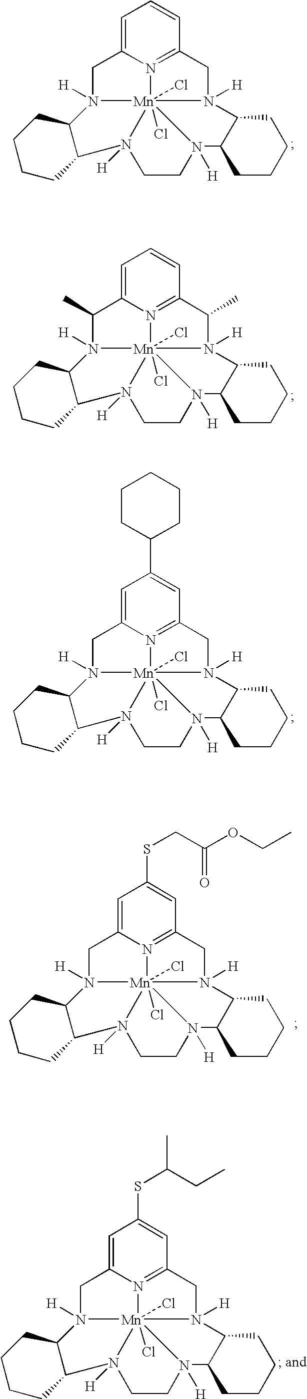

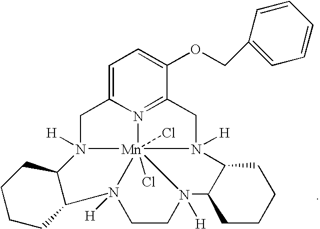

9. The composition of claim 6, wherein the composition is a solution or suspension for topical ophthalmic administration, for depot administration or for intraocular injection.

10. The composition of claim 9, wherein the concentration of the compound in the composition is from 0.001 to about 5% w/v.
